(19) United States
(12) Patent Application Publication
(10) Pub. No.: US 2007/0225810 A1
(43) Pub. Date: Sep. 27, 2007

(54) FLEXIBLE CAGE SPINAL IMPLANT

(76) Inventors: Dennis Colleran, North Attleboro, MA (US); Scott Schorer, Duxbury, MA (US)

Correspondence Address: CARR LLP (IST), 670 FOUNDERS SQUARE, 900 JACKSON STREET, DALLAS, TX, 75202, US

(21) Appl. No.: 11/690,677

(22) Filed: Mar. 23, 2007

Related U.S. Application Data
(60) Provisional application No. 60/785,195, filed on Mar. 23, 2006.
(60) Provisional application No. 60/825,089, filed on Sep. 8, 2006.

(51) Int. Cl. A61F 2/44; A61F 2/28

(52) U.S. Cl. 623/17.13; 623/23.41

(57) ABSTRACT

A implant is provided for placement in a space between boney structures. The implant may comprise a flexible section. The flexible section may be either the anterior side or the posterior side of the implant or both, among other sides. The flexible section or sections may comprise one or more orifices, cavities, or low modulus of elasticity materials among others. The flexible section or sections may facilitate a wider range of motion than otherwise possible for a spinal column comprising a Lumbar Interbody Fusion (LIF) device. Additionally, the anterior side comprising a flexible section may have a different modulus of elasticity than the posterior side comprising a flexible section. The difference may facilitate a wider range of responses from the implant to movement generated forces in at least two directions.

### CROSS-REFERENCE TO RELATED APPLICATIONS

This application relates to, and claims the benefit of the filing date of, co-pending U.S. Provisional Patent Application Ser. No. 60/785,195 entitled “FLEXIBLE CAGE SPINAL IMPLANT,” filed Mar. 23, 2006, the entire contents of which are incorporated herein by reference for all purposes. This application also relates to co-pending U.S. Provisional Application 60/825,089, entitled “OFFSET RADIUS LORDOSIS,” filed Sep. 8, 2006, and to U.S. patent application Ser. No. ______, entitled “INSTRUMENTS FOR DELIVERING SPINAL IMPLANTS” filed concurrently herewith, and to U.S. application Ser. No. 11/303,138, entitled “THREE COLUMN SUPPORT DYNAMIC STABILIZATION SYSTEM AND METHOD OF USE,” filed Dec. 16, 2005, the contents of which are incorporated herein by reference for all purposes.

### BACKGROUND OF THE INVENTION

### Field of the Invention

This disclosure relates to systems and methods for treating diseases of human spines, and more particularly, to interbody implant devices.

The human spine is a complex structure designed to achieve a myriad of tasks, many of them of a complex kinematic nature. The spinal vertebrae allow the spine to flex in three axes of movement relative to the portion of the spine in motion. These axes include the horizontal (e.g., bending either forward/anterior or aft/posterior), roll (e.g., lateral bending to either left or right side) and rotation (e.g., twisting of the shoulders relative to the pelvis).

The inter-vertebral spacing (between neighboring vertebrae) in a healthy spine is maintained by a compressible and somewhat elastic disc. The disc serves to allow the spine to move about the various axes of rotation and through the various arcs and movements required for normal mobility. The elasticity of the disc maintains spacing between the vertebrae during flexion and lateral bending of the spine, allowing room or clearance during the compressive movement of neighboring vertebrae. In addition, the disc enables relative rotation about the vertical axis of the neighboring vertebrae, allowing for the twisting of the shoulders relative to the hips and pelvis. Clearance between neighboring vertebrae maintained by a healthy disc is also important to enable the nerves from the spinal cord to extend out of the spine, between neighboring vertebrae, without being squeezed or impinged by the vertebrae.

In situations (e.g., based upon injury or otherwise) where a disc is not functioning properly, the inter-vertebral disc tends to over compress. With the over compression, pressure may be exerted on nerves extending from the spinal cord due to this reduced inter-vertebral spacing. Various other types of nerve problems may also be experienced in the spine, such as exiting nerve root compression in neural foramen, passing nerve root compression, and enervated annulus (i.e., where nerves grow into a cracked/compromised annulus, causing pain every time the disc/annulus is compressed), as examples. Many medical procedures have been devised to alleviate such nerve compression and the pain that results from the nerve pressure. Many of these procedures revolve around attempts to prevent the vertebrae from moving too close to each other by surgically removing an improperly functioning disc and replacing the disc with a lumbar interbody fusion (“LIF”) device. Although prior interbody devices, including LIF cage devices, may be effective at improving patient condition, these LIF cage devices may not provide the range of flexibility and support of a properly functioning disc.

It would be desirable to improve the flexibility of the LIF cage devices, while maintaining the high strength, durability and reliability, of the LIF cage device. A flexible LIF cage device may better enable a patient move about the various axes of rotation and through the various arcs and movements required for a normal range of mobility.

### SUMMARY

An embodiment of the present invention may comprise a flexibility enabling member on a section of an implant.

### BRIEF DESCRIPTION OF THE DRAWINGS

For a more complete understanding of this disclosure reference is now made to the following Detailed Description taken in conjunction with the accompanying drawings, in which:

FIG. 1 illustrates an oblique view of an embodiment of a flexible spinal implant designed to be inserted into an intervertebral space;

FIG. 2 illustrates a top view of the flexible spinal implant;

FIG. 3 illustrates an anterior view of the flexible spinal implant;

FIG. 4 illustrates a midline cross-sectional view of the flexible spinal implant;

FIG. 5 illustrates an anterior view of the flexible spinal implant, wherein a force is applied to the top portion of the implant;

FIG. 6A illustrates a side view of the flexible spinal implant, wherein a force is applied to the anterior portion of the implant;

FIG. 6B illustrates an alternative side view of the flexible spinal implant, wherein a force is applied to the posterior portion of the implant;

FIG. 7 illustrates an oblique view of the flexible spinal implant, wherein openings of the implant may be pushed out;

FIGS. 8A-D illustrate anterior views of some of the various embodiments of the flexible spinal implant;

FIG. 9 illustrates a sagittal view of the flexible spinal implant, wherein the implant is located between two adjacent vertebrae;

FIG. 10 illustrates an oblique view of a flexible spinal implant, wherein the implant is being injected with a material;

FIG. 11A illustrates a sagittal view of the flexible spinal implant, wherein the implant comprises a port for injecting a material; and

FIG. 11B illustrates a midline section view of the flexible spinal implant, wherein the implant comprises a port for injecting a material.

### DETAILED DESCRIPTION

In the following discussion, numerous specific details are set forth to provide a thorough understanding of the present invention. However, those skilled in the art will appreciate that the embodiments described in this disclosure may be practiced without such specific details. In other instances, well-known elements may have been illustrated in schematic or block diagram form in order not to obscure the present invention in unnecessary detail. Additionally, for the most part, details concerning well known features and elements may have been omitted inasmuch as such details are not considered necessary to obtain a complete understanding of the present invention, and are considered to be within the understanding of persons of ordinary skill in the relevant art.

### An Illustrative Embodiment

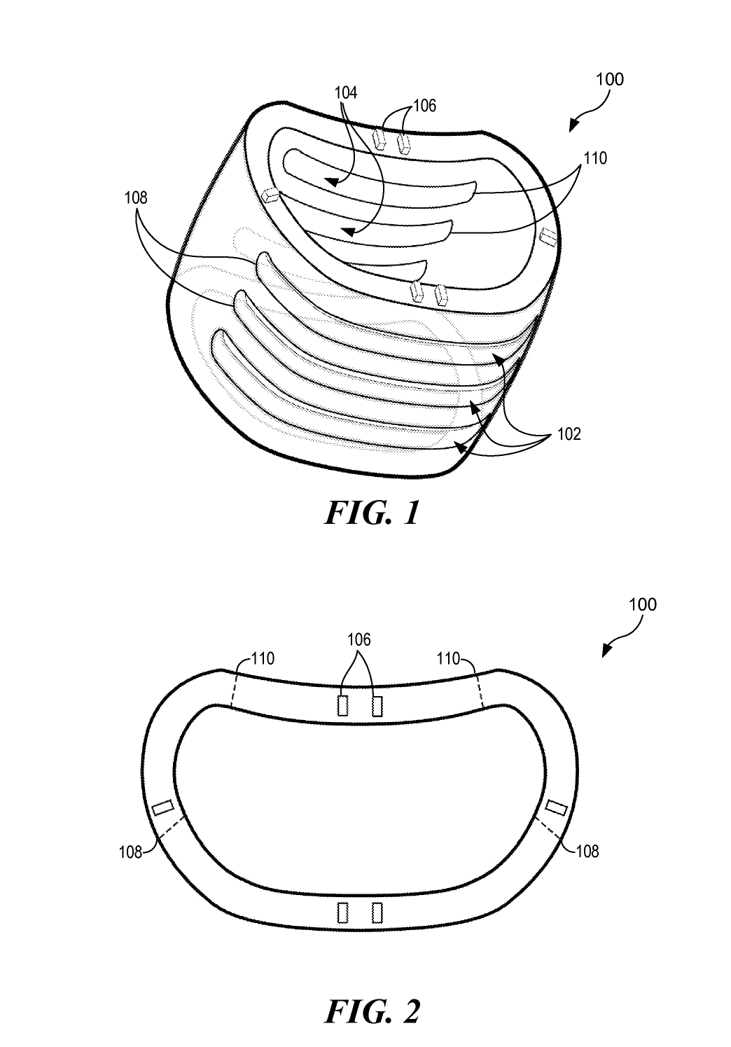

Turning now to the drawings, FIG. 1 shows an oblique view of an illustrative embodiment of a flexible spinal implant 100 configured according to at least a portion of the subject matter of the present invention and designed to be inserted into an intervertebral space. The flexible spinal implant 100 may have multiple flexural components 102 provided in an anterior surface of the implant 100. The flexible spinal implant 100 may also have multiple flexural components 104 provided in a posterior surface of the implant 100. These flexural components 102 and 104 may comprise empty space (e.g., voids, apertures, cavities, or no material) or they may be filled with a material having a lower modulus of elasticity than a surrounding portion of the implant 100. The flexural components 104 may be of a similar configuration to the flexural components 102, or they may be different. Additionally, all of the flexural components 102, 104 of an anterior or a posterior surface may comprise the same or different configurations. Although, multiple flexural components 102, 104 are shown in this illustrative embodiment of the present invention, a single flexural component 102, 104 may exist on an anterior and/or a posterior surface.

Multiple protrusions 106 may be located on the top surface and/or the bottom surface of the implant 100. In certain embodiments, these protrusions 106 may help to prevent the implant 100 from substantially moving within the intervertebral space. Although the protrusions 106 may be shown in FIG. 1 as being rectangular shaped, the protrusions 106 may not be limited to this configuration. Any geometric configuration may be used. In addition, an undulating surface may also provide the benefit of fixing the implant 100 in place without necessarily being a distinct protrusion. A single protrusion 106 on the top surface and/or the bottom surface of the implant 100 may also be used. The protrusions 106 may restrain the implant in a relatively fixed location by engaging the opposing surfaces of the endplates of adjacent vertebrae.

As shown in FIG. 1, in some embodiments the endpoints 108 of the anterior flexural components 102 may extend to the side surfaces of the implant 100. The endpoints 110 of the posterior flexural components 104 may be limited to the posterior surface of the implant 100. Accordingly, in multiple embodiments the anterior flexural components 102 and the posterior flexural components 104 may have a wide range of lengths, widths, and positions. These flexural components 102 and 104 may be configured to alter, reposition, or increase the flexibility of the spinal implant 100.

With multiple flexural components 102 on the anterior surface of the implant 100, the anterior surface of the implant 100 may exhibit an increased ability to resiliently deform when a force is applied to the anterior portion of the implant 100. Similarly, with multiple flexural components on the posterior surface of the implant 100, the posterior surface of the implant 100 may also exhibit an increased ability to resiliently deform when a force is applied to the posterior portion of the implant 100. Accordingly, the implant 100 may be able to provide support within the intervertebral space and also provide a range of flexibility when adjacent vertebrae exert a force on the implant 100. In certain embodiments, these flexural components 102 and 104 may provide flexibility through less material (e.g., through the use of a cavity, orifice, or a variable thickness of material), which may produce a lower modulus of elasticity, or through a lower modulus material (e.g., through the use of different heat treatments or material processing, or the substitution or addition of a separate material).

The implant 100 may be manufactured from a variety of biocompatible materials. For example, the implant 100 may be made from biocompatible plastics or metals such as PEEK(poly-ether-ether-ketone), carbon filled PEEK, titanium, or stainless steel, among others. The implant 100 may preferably comprise a sufficient level of strength to at least partially replace a supporting function of an intervertebral disc such that adjacent vertebrae may maintain a desired minimum amount of spacing between opposing surfaces. In some embodiments, the implant 100 may be made of metal, such as cobalt chrome, or titanium. In other embodiments, the implant 100 may be made of ceramic materials or a combination of both metal and ceramic materials, such as oxidized zirconium.

Turning now to FIG. 2, this figure illustrates a top view of the flexible spinal implant 100. Multiple protrusions 106 may be located on the top portion of the implant 100. As more easily seen in FIG. 2, in some embodiments the length of the anterior flexural components, which may be defined by the endpoints 108, may be longer than the length of the posterior flexural components, which may be defined by the endpoints 110. In this view, the endpoints 108 may be seen as extending to the sides of the implant 100 while the endpoints 110 may be confined to the posterior side surface of the implant 100. However, the locations and separations of the various endpoints 108, and 110 may not be limited to this illustrative embodiment.

The implant 100 may be a substantially oval-shape with a relatively empty center. This oval-shape of the implant 100 may correspond to the shape of the intervertebral disc. This empty center of the implant 100 may be filled with cadaveric bone, autologous bone, bone slurry, bone morphogenic protein (“BMP”) or a similar material. These types of materials may help with tissue growth within the intervertebral space. In some embodiments, openings created by the openings 102 and 104 may further help with tissue growth by allowing the material to seep into the intervertebral space. The illustrative embodiment is shown with a relatively consistent wall thickness. However, depending upon the flexibility configuration, the wall thickness may vary around the perimeter of the implant 100.

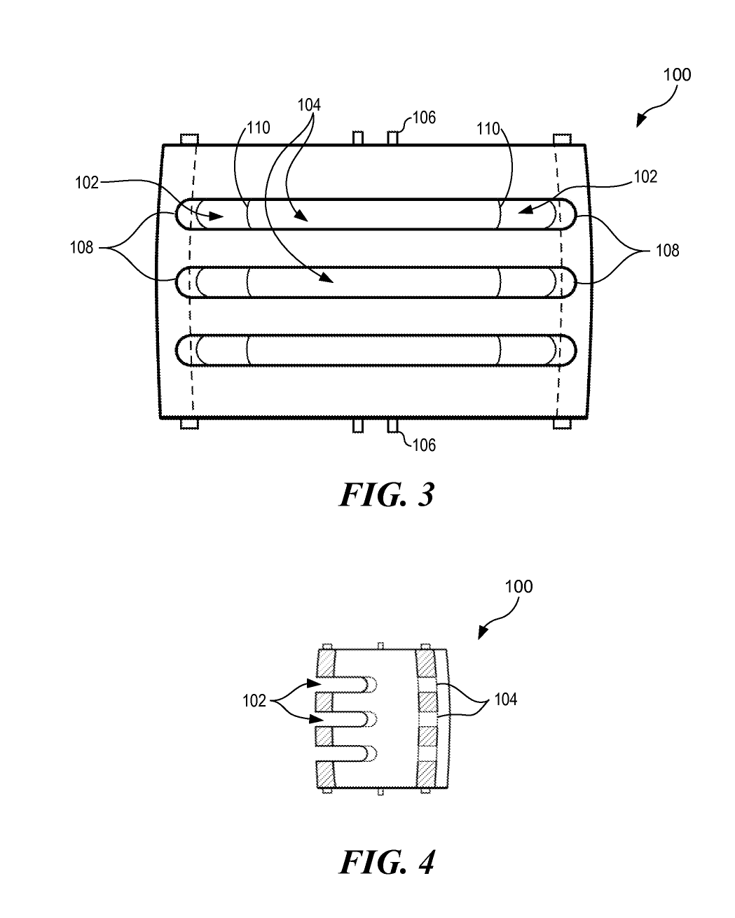

Referring now to FIG. 3, this figure illustrates an anterior view of the flexible spinal implant 100. As stated previously, in certain embodiments the anterior openings 102 may extend further in length than the posterior openings 104 (the posterior openings 104 are seen through the anterior openings 102 in this figure). Accordingly, from an anterior view the endpoints 110 of the posterior openings 104 may be visible because the endpoints 108 of the anterior openings 102 may extend to the side portions of the implant 100. The anterior openings 102 are shown as being approximately the same number and overall design as the posterior openings 104 as an example of one amongst many embodiments. The protrusions 106 are shown as existing on both the top surface and the bottom surface of the implant 100 in this representation of an exemplary embodiment.

Turning now to FIG. 4, this figure shows a midline cross-sectional sagittal view of the flexible spinal implant 100. As seen in this drawing, in certain embodiments the anterior openings 102 may extend to the side portions of the implant 100, while the posterior openings 104 may not extend to the side portions of the implant 100. In addition, the top and bottom surfaces may be substantially parallel in the absence of an applied force to the implant 100.

However, some embodiments of the implant (not shown) may be configured such that the top or bottom surfaces may be at an angle to each other in an unloaded condition. These implants may help to restore or recreate a lordosis angle (or other angle) of a human spine. In addition, both of the top and bottom surfaces of the implant may be at an angle relative to a horizontal midline of the implant in an unloaded condition. Alternatively, in certain embodiments (not shown), the top and/or bottom surfaces may be formed from a curved or compound curved surface, instead of the relatively straight line configurations shown in the figure. These implants may also help to restore or recreate a lordosis angle (or other angle) of a human spine. In addition, the contoured top and bottom surfaces (i.e., superior and inferior surfaces) may conform more closely to the concave end plates of the adjacent vertebra. More particularly, the compound curved surfaces may be created by offsetting the radii used to machine the top and bottom (i.e., bearing) surfaces of the implant.

Further, the cross-sections are shown in FIG. 4 with relatively straight line configurations to aid in simplifying the figures. Although an embodiment of the current invention may be formed as shown, the implant may not be limited to such a configuration. The cross-sections may comprise curved, angular, arcuate, and other configurations able to alter the flexibility of the implant 100. Additionally, all of the anterior openings 102 and the posterior openings 104 are shown as establishing communication between the interior and the exterior of the implant 100. As stated previously, in some embodiments, the anterior openings 102 and/or the posterior openings 104 may extend only partially through the walls of the implant 100.

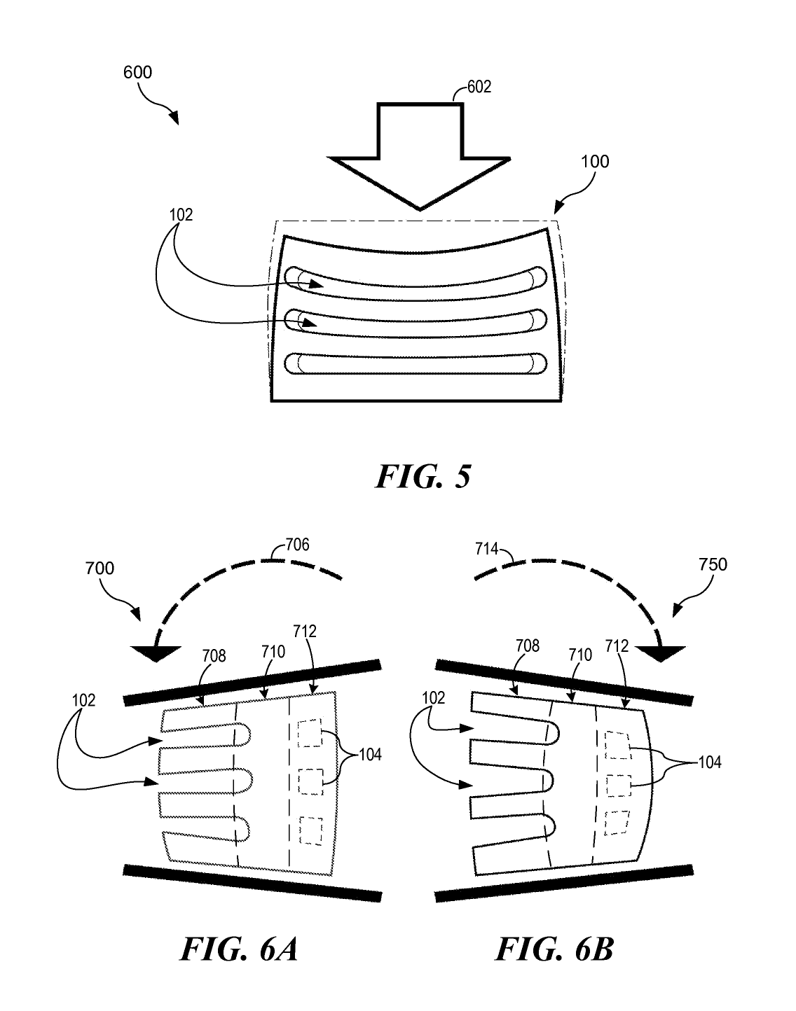

Referring now to FIG. 5, this figure illustrates an anterior view of the flexible spinal implant 100 (shown in broken lines), wherein a force 602 is applied to the top portion of the implant 100. The force 602 applied to the top portion of the implant 100 may cause the implant 100 to deform or compress into a form of an implant 600 (actual deformation may be exaggerated in this figure for the purposes of illustration). As shown in FIG. 5, the anterior openings 102 may also compress, enabling the top surface of the implant 600 to move closer to the bottom surface of the implant 600. The deformation of the implant 600 may enable a larger range of motion for a spinal column in which the implant 600 has been inserted. The deformation is shown as being larger in the central section than at the sides of the implant 600. This may be due in part to the increased stiffness of the sides of the implant 600 due to a relatively smaller quantity of openings. Although the posterior openings 104 (FIG. 3) may not be visible in FIG. 5, these openings 104 may exhibit a similar type of compression in response to a force applied to the implant 100.

Turning now to FIG. 6A, this figure shows a side view of a spinal implant 700 in which a force 706 has been applied to an anterior portion of the implant 700. When a force 706 is applied to an implant (e.g., such as illustrated in FIG. 4), the anterior openings 102 may compress as described with reference to FIG. 5. In addition, since the force 706 may be applied primarily to the anterior portion of the implant 700, the posterior openings 104 may expand. This corresponding behavior between the openings 102 and the openings 104 may be attributed at least in part to the additional flexibility provided by the openings 102 and the openings 104 (the deformation may be exaggerated for the purposes of illustration).

Accordingly, an area comprising the anterior openings 102 may be defined as a first flex-zone 708 of the implant 700, while an area comprising the posterior openings 104 may be defined as a second flex-zone 712 of the implant 700. The first flex-zone 708 may flexibly contract while the second flex-zone 712 may flexibly expand. However, in the event of a relatively uniform force applied to the top surface of the implant 700, both the first flex-zone 708 and the second flex-zone 712 may be flexibly contracted or expanded, to either the same or differing degrees, depending upon the quantities and configurations of the anterior openings 102 and the posterior openings 104.

The middle portion of the implant 700, which may comprise the side walls, may be defined as a low-flex-zone 710 of the implant 700. The low-flex-zone 710 may provide a more consistent level of support for two adjacent vertebrae, while the flex-zones 708 and 712 may provide additional flexibility. This additional flexibility may provide an additional range of motion with respect to the two adjacent vertebrae. The low-flex-zone 710 may help to prevent excessive vertical compression and consequential damage to nerve endings passing between the two adjacent vertebrae. The relatively stronger low-flex-zone 710 may also provide a more stable platform for the flex-zones 708 and 712.

Referring now to FIG. 6B, this figure illustrates an alternative side view of a flexible spinal implant 750 in which a force 714 has been applied to a posterior portion of the implant 750. When a force 714 is applied to an implant (e.g., such as illustrated in FIG. 4), the posterior openings 104 may contract and the anterior openings 102 may expand. As stated previously, the area comprising the anterior openings 102 and the area comprising the posterior openings 104 may be described as the flex-zones 708 and 712, respectively. The middle portion of the implant 750, which may comprise the side walls, may be described as the low-flex-zone 710 of the implant 750.

As shown in FIGS. 6A and 6B, there may be at least two degrees of motion for an implant 700, 750 depending upon the direction and location of the applied force. The motion illustrated in an embodiment of the present invention may allow for more natural movement of a spinal column and may begin to replace at least a portion of the functionality of a collapsed intervertebral disc. Additionally, the openings 102 and 104 may function to control motion during both expansion and contraction of an implant 700, 750.

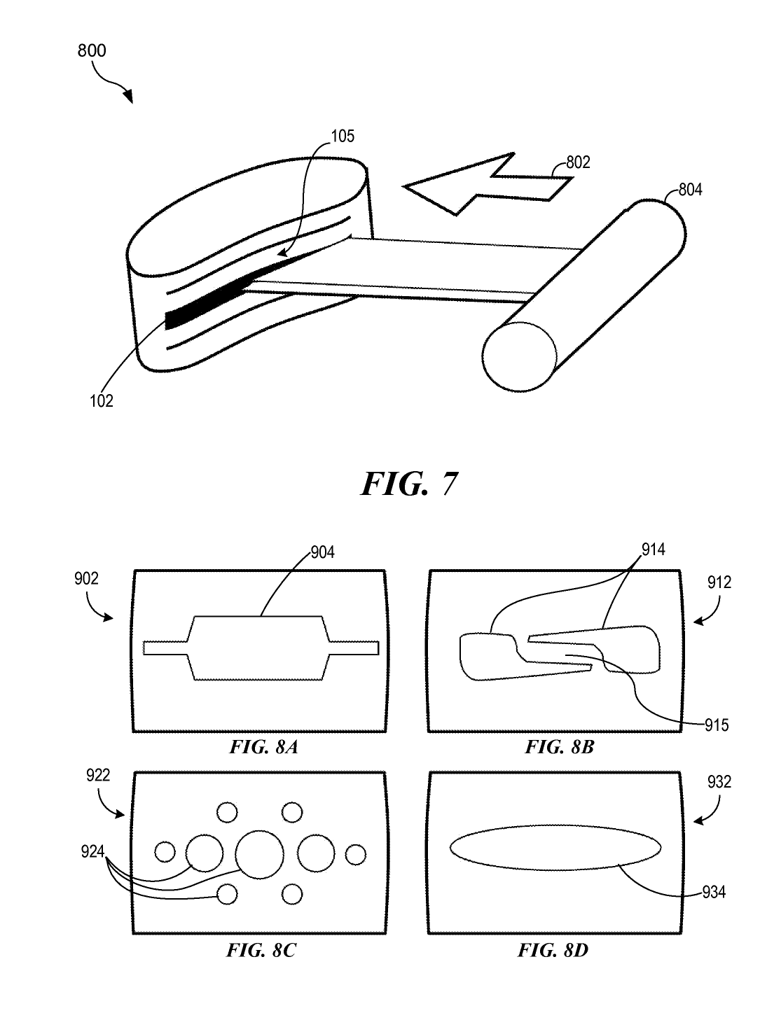

Turning now to FIG. 7, this figure shows an oblique view of an embodiment of a flexible spinal implant 800 in which the openings 102 of the implant 800 may be pushed out or removed. In certain embodiments, the implant 800 may have one or more removable members 105 retained within the implant 800 through the use of perforated dividers, interlocking features, friction forces, threaded fasteners, and adhesive forces, among others. The removable members 105 may be detached in response to a force 802 applied to the anterior or posterior portion of the implant 800. Accordingly, a tool 804 may be utilized to apply a force 802 to the implant 800 and produce an opening 102, by detaching the removable members.

This feature may enable a physician to adjust the flexibility of the anterior or posterior portion of a standard or common implant 800 to be adapted to the specific needs of a patient or a specific requirements of a portion of a patient's spine. The removable portions 105 may be removed prior to insertion of the implant 800 within a patient's body. However, there may be situations in which a range of motion of a patient may be adjusted via the removable members 105 after insertion. Additionally, the implant 800 is shown as configured with removable members 105. However, the flexibility of the implant 800 may be also be adjusted through the insertion of members with appropriate degrees of flexibility into openings 102. In some embodiments, the distraction height that the implant 800 provides may be increased by placing appropriate inserts into the openings 102. Consequently, the flexibility of a portion of a standard or common implant 800 may be increased or decreased (i.e., modified) through the removal of removable members 105 and/or insertion of other inserts into the openings 102.

Referring now to FIG. 8A, this figure illustrates an anterior view of an embodiment of the flexible spinal implant 902. In one example amongst many of an embodiment, the implant 902 may comprise a single opening 904. The opening 904 for example, may be irregularly shaped, symmetrical, or asymmetrical, in order to provide additional flexibility to the anterior portion (for example) of the implant 902. The overall design configuration for the opening 904 may be determined based upon results from finite element analysis for example.

Turning now to FIG. 8B, this figure shows an anterior view of another alternative embodiment of the flexible spinal implant 912. In one example of an embodiment of the present invention, the implant 912 may comprise two corresponding openings 914. These corresponding openings 914 may provide additional flexibility to the anterior portion (for example) of the implant 912. As seen in FIG. 8B, the two corresponding openings 914 may be configured to create an interconnecting member 915 located there between. The interconnecting member 915 may provide an additional degree of resiliency for the anterior portion of the implant 912. While the interconnecting member 915 may be shown as being integral to the anterior portion of the implant 912, other resilient members such as springs, compressible material, and others may be used to provide the additional degree of resiliency.

Referring now to FIG. 8C, this figure illustrates an anterior view of another alternative embodiment of the flexible spinal implant 922. In one illustrative embodiment, the implant 922 may comprise multiple circular or other configurations of openings 924. As shown in this example, these cylindrical openings 924 may provide additional flexibility to the anterior portion (for example) of the implant 922. Cylindrical openings 924 may be easily created in the anterior portion of the implant 922 through the use of drills or cores during molding for example. As with the illustrative embodiment discussed along with FIG. 7, the numbers, sizes, and placements, of the openings 924 may be made in a more common, generic implant according to the requirements of the patient.

Turning now to FIG. 8D, this figure shows an anterior view of an alternative embodiment of the flexible spinal implant 932. In one example of an embodiment, the implant 932 may comprise a single oval-shaped opening 934. The oval-shaped opening 934 may provide additional flexibility to the anterior portion (for example) of the implant 932. A large relatively smooth opening such as the opening 934 may reduce local areas of stress concentration within the implant 932.

Additional embodiments of the anterior portion of an implant 100 are within the scope of this disclosure. This disclosure should not be limited to the embodiments shown in FIGS. 8A-8D. In addition, the embodiments shown in FIGS. 8A-D and other additional alternative embodiments of openings may be applied to the posterior portion or side portions of an implant 100. The other embodiments may be applied singly, in multiple numbers, or in combinations without limit as long as the flexibility and strength of an implant 100 are maintained at desired levels.

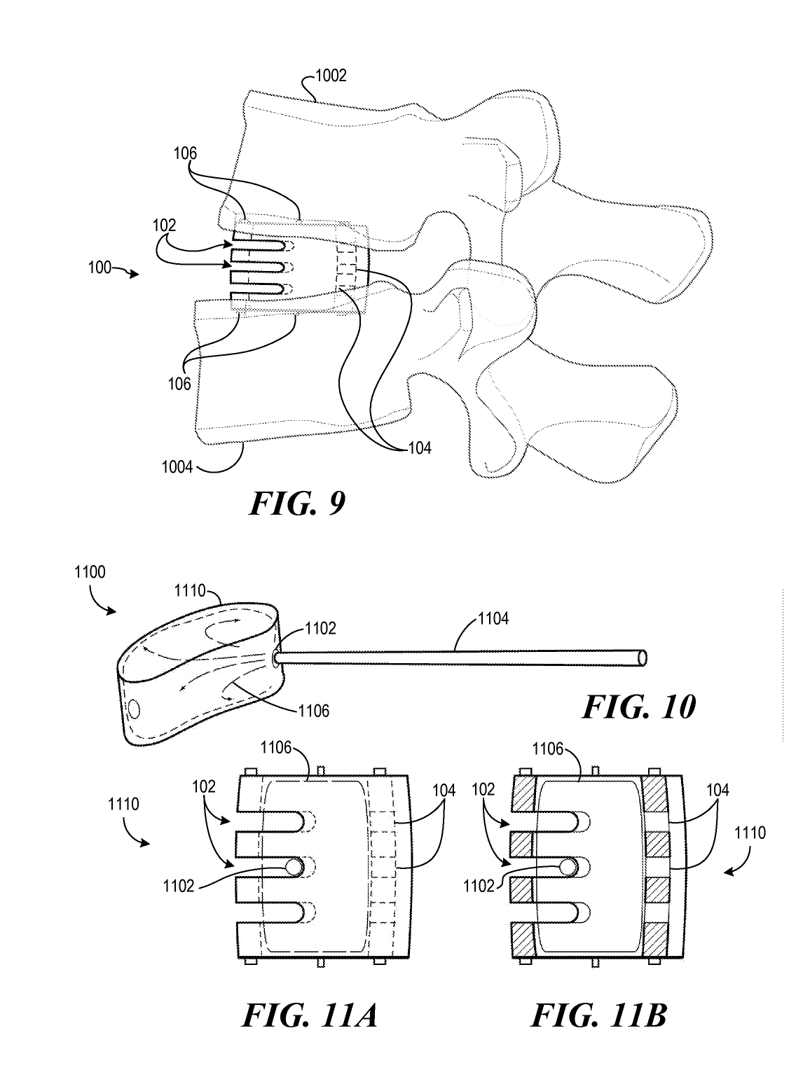

Referring now to FIG. 9, this figure illustrates a sagittal view of the flexible spinal implant 100 in which the implant 100 is located between two adjacent vertebrae 1002 and 1004. As shown in FIG. 9, the implant 100 may be placed in an intervertebral space. In this position, the flexible spinal implant 100 may function similarly to an intervertebral disc by providing both support and flexibility. Accordingly, anterior openings 102 and posterior openings 104 may provide an appropriate amount of flexibility to the implant 100.

Protrusions 106 may help to prevent the implant 100 from significantly moving within the intervertebral space relative to the two adjacent vertebrae 1002 and 1004. The protrusions 106 may be located on the top and bottom surface of the implant 100 and engaged with the opposing surfaces of the two adjacent vertebrae 1002 and 1004.

In certain embodiments the implant 100 may be configured as a dynamic device, such as a partial disc replacement (PDR). The implant 100 may be used to stabilize adjacent vertebrae as the spine moves in various directions. A dynamic stabilization device may be used in conjunction with the implant 100 as part of a three column support dynamic stabilization system as is described in more detail in co-pending U.S. application Ser. No. 11/303,138, entitled “THREE COLUMN SUPPORT DYNAMIC STABILIZATION SYSTEM AND METHOD OF USE,” filed Dec. 16, 2005, and incorporated herein by reference for all purposes.

Turning now to FIG. 10, this figure shows an oblique view of a flexible spinal implant 1110 in which the implant 1110 is being injected with a material 1106. This material 1106 may be injected in situ. In one embodiment, the implant 1110 may have a port 1102. An insertion tube 1104 may couple to the port 1102 such that a material 1106 may be injected into the interior of the implant 1110. This material 1106 may be utilized to provide additional support or flexibility, or to enhance tissue growth within the intervertebral space. Accordingly, materials such as cadaveric bone, autologous bone, bone slurry, BMP, or other similar material, may enhance tissue growth within the intervertebral space. In some embodiments, a separate container or walls may be provided to contain the material within the interior of the implant 1110.

Referring now to FIG. 11A, this figure illustrates a sagittal view of the flexible spinal implant 1110 in which the implant 1110 comprises the port 1102 for injecting the material 1106. The port 1102 may be located in any of the anterior openings 102 and the posterior openings 104, or the port 1102 may be located in an opening configured specifically for the port 1102. The material 1106 may be injected into the implant 1110 via this port 1102. The material 1106 may fill the center portion of the implant 1110 as shown in FIG. 11A. In addition, only two ports are shown in FIG. 10 and only one port 1102 is visible in FIG. 11A, however, a single port or a plurality of ports 1102 may be provided in the implant 1110. Further, although a separate port 1102 may be described for inserting the material 1106, the material 1106 may be inserted through an existing anterior and/or posterior opening 102 and 104.

Turning now to FIG. 11B, this figure shows a midline cross-sectional view of the flexible spinal implant 1110, in which the implant 1110 comprises a port 1102 for injecting the material 1106. The material 1106 may be injected into the implant 1110 via this port 1102. The material 1106 may fill the center portion of the implant 1110 as shown in FIG. 11B. As previously stated with regard to FIG. 4, in certain embodiments the anterior openings 102 may extend to the side portions of the implant 1110, while the posterior openings 104 may not extend to the side portions of the implant 1110. In addition, the top and bottom surfaces may be substantially parallel in the absence of an applied force to the implant 1110.

The cross-sections are shown with relatively straight line configurations for the purposes of illustration. The cross-sections may comprise curved, angular, arcuate, and other configurations able to alter the flexibility of the implant 1110. Additionally, all of the anterior openings 102 and the posterior openings 104 are shown as establishing communication between the interior and the exterior of the implant 1110. In some embodiments, the anterior openings 102 and/or the posterior openings 104 may extend only partially through the walls of the implant 1110. The insertion port 1102 may establish communication between the interior and the exterior of the implant 1110. The insertion port 1102 may further comprise corresponding engagement surfaces for locating an insertion tube 1104 (FIG. 10) in addition to one way valves or devices necessary to facilitate the insertion of material 1106 into the interior of the implant 1110.

It is understood that multiple embodiments can take many forms and designs. Accordingly, several variations of these embodiments may be made without departing from the scope of this disclosure. Having thus described specific embodiments, it is noted that the embodiments disclosed are illustrative rather than limiting in nature. A wide range of variations, modifications, changes, and substitutions are contemplated in the foregoing disclosure. In some instances, some features may be employed without a corresponding use of the other features. Many such variations and modifications may be considered desirable by those skilled in the art based upon a review of the foregoing description of embodiments.

1. A flexible implant configured to facilitate at least some relative movement between neighboring boney structures comprising:
a first section at least slightly curved inward toward an interior of the implant and comprising at least one flexible member and configured to facilitate at least some amount of expansion and contraction of the flexible implant;
a second section spaced from the first section, comprising at least one flexible member and configured to facilitate at least some amount of expansion and contraction of the flexible implant; and
wherein the first section comprises an overall modulus of elasticity not equal to the overall modulus of elasticity of the second section, whereby the first and second sections provide different resilient support to neighboring boney structures.

2. The flexible implant of claim 1 wherein the at least one flexible member in the first section comprises a plurality of flexible members.

3. The flexible implant of claim 2 wherein the at least one flexible member in the second section comprises a plurality of flexible members.

4. The flexible implant of claim 1 wherein the at least one flexible member in at least one of the first section and the second section comprises an orifice.

5. The flexible implant of claim 4 wherein the orifice comprises a slot.

6. The flexible implant of claim 4 wherein the orifice comprises a cylindrical hole.

7. The flexible implant of claim 1 wherein the overall modulus of elasticity of the first section and the overall modulus of elasticity of the second section are not equal to an overall modulus of elasticity of a remaining portion of the flexible implant.

8. The flexible implant of claim 1 wherein a configuration of at least one of the flexible members in at least one of the first section and the second section is not the same as a configuration of a remaining portion of flexible members.

9. A flexible implant comprising:
a first section;
a second section spaced from the first section;
wherein each of the first section and the second section comprise a flexible member, whereby the first section comprises a first nominal modulus of elasticity and the second section comprises a second nominal modulus of elasticity;
wherein the first nominal modulus of elasticity and the second nominal modulus of elasticity are different than a remaining nominal modulus of elasticity for a remaining portion of the flexible implant; and
wherein the first nominal modulus of elasticity is not equal to the second nominal modulus of elasticity, whereby the implant is able to provide different responses to forces due to at least two types or directions of movement.

10. A flexible implant comprising:
a first section at least slightly curved inward toward an interior of the implant;
a second section at least slightly curved outward away from the interior of the implant;
at least one flexible member in each of the first section and the second sections and configured to resiliently respond to at least some movement of the flexible implant in at least two directions of motion; and
wherein the first section is more resiliently deformable than the second section.

11. The flexible implant of claim 10, wherein the first section comprises a first nominal modulus of elasticity resulting at least in part from the at least one flexible member in the first section;
wherein the second section comprises a second nominal modulus of elasticity resulting at least in part from the at least one flexible member in the second section; and
wherein the first nominal modulus of elasticity is not equal to the second nominal modulus of elasticity.

12. The flexible implant of claim 10, wherein the first section comprises a first nominal modulus of elasticity due at least in part to the at least one flexible member in the first section;
wherein the second section comprises a second nominal modulus of elasticity due at least in part to the at least one flexible member in the second section; and
wherein the second nominal modulus of elasticity is greater than the first nominal modulus of elasticity.

13. The flexible implant of claim 12, wherein the at least one flexible member comprises an orifice.

14. The flexible implant of claim 13, wherein the orifice is an elongated slot.

15. The flexible implant of claim 13, wherein the orifice is cylindrically shaped.

16. The flexible implant of claim 12, wherein the at least one flexible member comprises a resilient material.

17. The flexible implant of claim 12, wherein the at least one flexible member comprises a depression.

18. The flexible implant of claim 10, further comprising:
a first abutment surface for contacting a first boney surface;
a second abutment surface for contacting a second boney surface; and
wherein at least one protrusion is provided on at least one of the first abutment surface and the second abutment surface.

19. The flexible implant of claim 18, wherein at least one protrusion is provided on each of the first abutment surface and the second abutment surface.

20. The flexible implant of claim 18, wherein the at least one protrusion comprises a plurality of protrusions.

21. The flexible implant of claim 19, wherein the at least one protrusion on each of the first abutment surface and the second abutment surface comprises a plurality of protrusions.

22. A method for adjusting a modulus of elasticity for an implant, comprising:
determining a modulus of elasticity range for at least a first portion of the implant in at least one direction of motion; and
adjusting the modulus of elasticity range of the first portion of the implant, comprising the steps of:
removing one or more removable sections of the implant from the first portion of the implant such that the modulus of elasticity is within the determined modulus of elasticity range; or
inserting a spacing member into one or more openings in the first portion of the implant.

23. The method of claim 22, wherein the step of adding an insert further comprises distracting the first portion of the implant by inserting a spacing member into at least one of the one or more openings of the first portion.
